(19) United States
(12) Patent Application Publication
(10) Pub. No.: US 2025/0135003 A1
(43) Pub. Date: May 1, 2025

(54) SCINTILLATOR, POWDERS HAVING SCINTILLATORS, SUSPENSION HAVING SCINTILLATORS AND METHOD OF FORMING SCINTILLATORS

(71) Applicants: UNIVERSITA' DEGLI STUDI DI MILANO BICOCCA, MILANO (MI) (IT); BAR-ILAN UNIVERSITY, RAMAT GAN (IL)

(72) Inventors: Angelo MONGUZZI, Milano (MI) (IT); Valeria SECCHI, Milano (MI) (IT); Marcello CAMPIONE, Milano (MI) (IT); Shai RAHIMIPOUR, RAMAT GAN (IL); Sudipta SENAPATI, RAMAT GAN (IL)

(73) Assignees: UNIVERSITA' DEGLI STUDI DI MILANO - BICOCCA, MILANO (MI) (IT); BAR-ILAN UNIVERSITY, RAMAT GAN (IL)

(21) Appl. No.: 18/909,262

(22) Filed: Oct. 8, 2024

(30) Foreign Application Priority Data
Oct. 27, 2023 (IT) 102023000022626

(51) Int. Cl. A61K 41/00; A61N 5/06

(52) U.S. Cl. CPC A61K 41/0071; A61K 41/0057; A61K 41/0061; A61N 5/062

(57) ABSTRACT

There is described a scintillator for electromagnetic radiation therapy comprising a nanotube being functionalized with a plurality of singlet oxygen photosensitizers configured to form singlet oxygen upon excitation by means of an electromagnetic radiation; and a plurality of ligands configured to stabilize the scintillator within a fluid comprising water and/or to minimize interaction with biomolecules present within the fluid.

### CROSS-REFERENCE TO RELATED APPLICATIONS

This patent application claims priority from Italian patent application no. 102023000022626 filed on Oct. 27, 2023, the entire disclosure of which is incorporated herein by reference.

### TECHNICAL FIELD

The present invention relates to scintillators for electromagnetic radiation therapy.

Additionally, the present invention relates to a composite for electromagnetic radiation therapy and comprising a plurality of scintillators.

Moreover, the present invention relates to a suspension or solution for electromagnetic radiation therapy and comprising a plurality of scintillators.

Advantageously, the present invention also relates to the use of scintillators in the treatment of diseases.

Moreover, the present invention also relates to a method of forming scintillators.

### BACKGROUND ART

Alzheimer's disease (AD) is a neurodegenerative disorder that affects memory, cognition, and behavior, and is the most common cause of dementia. As the population ages, the prevalence of AD is increasing, and there is an urgent need for effective treatments. AD is a progressive degenerative disease that induces heavy disability with no cure.

As represented in FIG. 1, misfolding of amyloid-β (Aβ) and tau proteins lead to the formation of Aβ oligomers 1, Aβ fibrils 2 and neurofibrillary tangles, which accumulate in the brain in pathogenic pathways leading to synaptic loss and selective neuronal cell death. Rich in cross-sheet conformations, soluble Aβ oligomers 1 are suspected to be more deleterious than Aβ fibrils 2 and plaques and they appear prior to tau aggregation and clinical symptoms. Therefore, soluble Aβ oligomers 1 have been recently tested as potential targets for the development of AD-specific drugs, with the aim to stop the decline of the patient's cognitive functions. Indeed, the monoclonal antibodies Aducanumab and Lecanemab have been recently approved by the Food and Drug Administration (FDA) for early AD treatment. However, a controversy over efficacy and side effects, such as microhemorrhages and brain swelling, due to administration of such monoclonal antibodies highlight the need for improvements.

In search of an alternative and non-invasive therapy, singlet oxygen (SO) generated by suitable photosensitizers and visible light were demonstrated to inhibit Aβ aggregation and toxicity thus achieving a peculiar photodynamic therapeutic effect. However, this strategy is limited by the penetration of the UV-visible photons through deep tissues and the skull.

Therefore, a need is felt in the field for improvements and/or new approaches and means to treat diseases such as AD or to at least slow down their progression.

### DISCLOSURE OF INVENTION

The present invention aims to provide improved means for electromagnetic radiation therapy of a disease, and in particular AD treatment.

According to the present invention, there is provided a scintillator as described herein and set forth in the appended claims.

Preferred embodiments are claimed in the dependent claims.

According to the present invention there is also provided a composite as described herein.

Additionally, according to the present invention there is also provided a suspension or solution as described herein.

According to the present invention there is also provided a method as described herein.

Finally, according to the preset invention there is also provided the use of scintillators as described herein.

### BRIEF DESCRIPTION OF THE DRAWINGS

A non-limiting embodiment of the present invention will be described by way of example with reference to the accompanying drawings, in which:

FIG. 1 schematically illustrates the progression of Alzheimer's disease;

FIG. 2 schematically illustrates the treatment of Alzheimer's disease by relying on scintillators according to the present invention, with parts removed for clarity;

FIG. 3A to 3C schematically illustrates the formation of scintillators according to the present invention, with parts removed for clarity; and

FIG. 4 illustrates Atomic force microscope (AFM) images of Aβ alone (left) and treated by the use of scintillators according to the present invention and following exposure to soft X-ray (right).

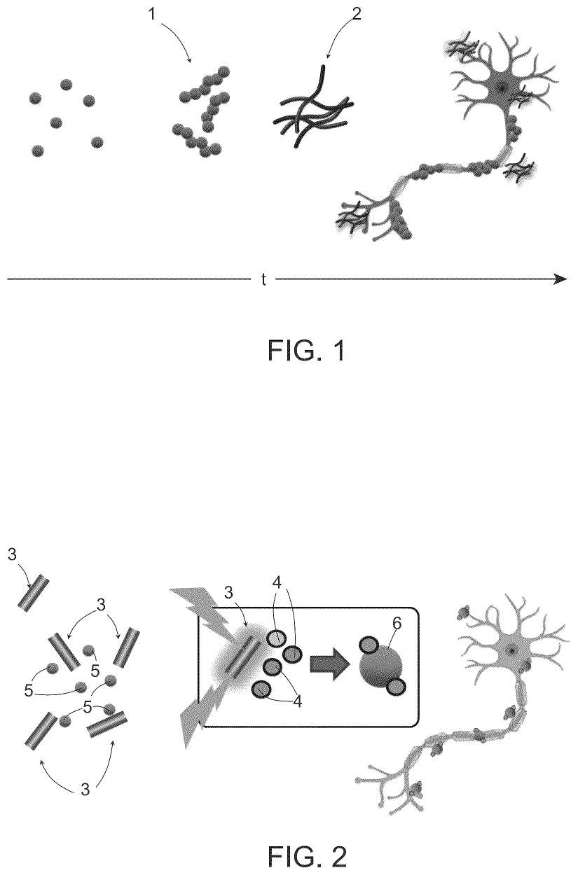

### BEST MODES FOR CARRYING OUT THE INVENTION

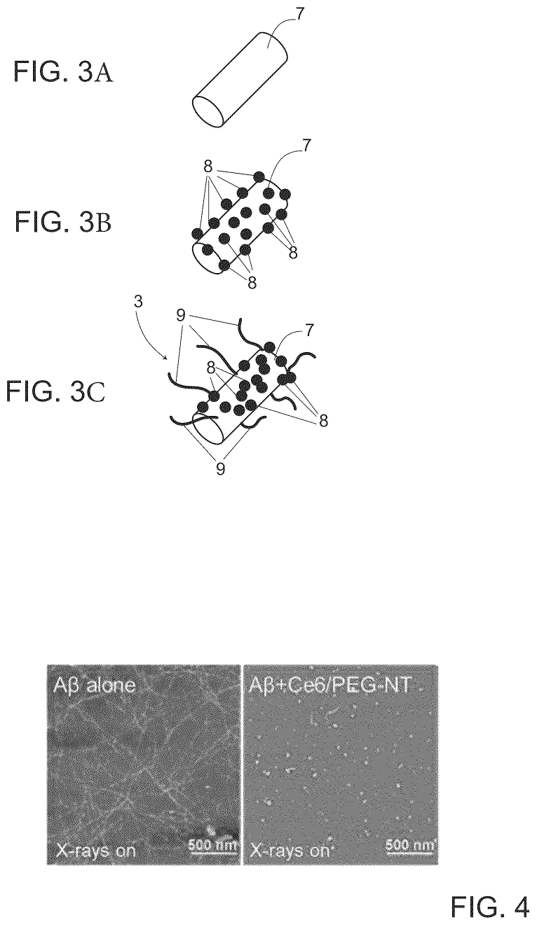

Number 3, as shown in FIGS. 2 and 3c, indicates as a whole a scintillator according to the present invention.

Scintillator 3 is configured to be used in electromagnetic radiation therapy, more specifically in radiotherapy, e.g. for the treatment of degenerative diseases such as Alzheimer's disease (AD).

More specifically, scintillators 3 may be configured to selectively interact with Aβ aggregates, such as Aβ oligomers 1, and upon radiation with electromagnetic radiation such as an ionizing radiation (e.g. X-ray radiation), may inhibit the formation of the neurotoxic Aβ aggregates, such as Aβ oligomers 1.

With particular reference to FIG. 2, scintillators 3 may be configured to form singlet oxygen 4 upon radiation with electromagnetic radiation, such as an ionizing radiation (e.g. X-ray radiation).

The Applicant has observed (in vitro and in vivo) that the exposure of scintillators 3 to electromagnetic radiation, e.g. to an ionizing radiation (such as X-ray radiation) allows to reduce the formation of Aβ oligomers 1 and their toxicity, which inhibits the progression of AD.

In more detail, scintillators 3 are configured to form singlet oxygen 4 when being irradiated with the electromagnetic radiation, e.g. to an ionizing radiation (such as X-ray radiation), and singlet oxygen 4, again, is configured to oxidize Aβ monomers 5 resulting in the formation of oxidized Aβ monomers 6.

In particular, scintillators 3 allow to be used in the inhibition of the formation of Aβ aggregates, such as Aβ oligomers 1.

With reference to FIGS. 3A to 3C, scintillator 3 comprises a nanotube 7 being functionalized with a plurality of singlet oxygen photosensitizers 8 configured to form singlet oxygen 4 upon excitation by means of the electromagnetic radiation, such as the ionizing electromagnetic radiation (e.g. X-ray radiation), and a plurality of ligands 9 configured to stabilize scintillator 3 and/or to increase solubility of scintillator 3 within a fluid, more specifically a liquid, comprising water and/or to minimize non-specific interactions with biomolecules present within the fluid.

In more detail, nanotube 7 may have a length ranging between 50 nm to 1000 nm and/or a diameter ranging between 50 nm to 130 nm.

In further detail, nanotube 7 may consist of and/or comprise a biocompatible material which allows nanotube 7 to be taken up by biological tissue and/or cells and/or to be able to penetrate the blood brain barrier (BBB), e.g. in particular in pathological conditions.

According to some possible embodiments, nanotube 7 may comprise and/or consist of magnesium silicate (Mg3(Si2O5)(OH)4) and/or may comprise alternating and wrapped layers of silica and brucite.

According to some possible embodiments, nanotube 7 may have an external surface, e.g. being brucitic with a positive ζ-potential, which may allow functionalization by means of ion self-assembly.

According to some possible embodiments, ligands 9 and photosensitizers 8 may be connected to the external surface of nanotube 7.

More specifically, ligands 9 and/or photosensitizers 8 may comprise respective active groups being connected to the external surface of nanotube 7.

In more detail, photosensitizers 8 may be negatively charged, e.g. allowing to immobilize photosensitizers 8 to nanotube 7 by means of ionic self-assembly.

According to some possible non-limiting embodiments, each photosensitizer 8 may comprise and/or consist of chlorin and/or chlorin e6 (Ce6-(17S,18S)-18-(2-carboxyethyl)-20-(carboxymethyl)-12-ethenyl-7-ethyl-3,8,13,17-tetramethyl-17,18,22,23-tetrahydroporphyrin-2-carboxylic acid).

In more detail, Ce6 exhibits a minimal toxicity, high stability, and high aqueous solubility and is, e.g. clinically approved for the treatment of lung cancer.

The Applicant has found that Ce6 as the chosen photosensitizer 8 is particularly efficient in the selective treatment of Aβ monomers 5 for avoiding the formation of Aβ aggregates.

For example, the Applicant has observed that the single oxygen 4 produced by the excited photosensitizers 8 (e.g. by excited Ce6) allows to oxidize the Histidine residues in Aβ monomers 5 and to generate cross-linked aggregates that hinder further aggregation.

Alternatively or in addition, photosensitizers 8 may comprise and/or consist of meso-tetra(4-sulfonatophenyl)-porphyrin (H2TPPS4-) and/or Rose Bengal (RB; 4,5,6,7-tetrachloro-2′,4′,5′,7′-tetraiodofluorescein) and/or porphyrin IX.

According to some possible embodiments, ligand 9 may be an inert ligand.

According to some possible embodiments, ligand 9 may be configured to stabilize scintillator 3 in the fluid (e.g. a biological liquid).

In particular, ligand 9 guarantees that scintillator 3 remains in suspension within the fluid (e.g. the biological liquid).

According to some possible embodiments, ligand 9 may comprise a respective functional group configured to connect ligand 9 to nanotube 7.

According to some preferred non-limiting embodiments, ligand 9 may be chosen from the group of polyols such as polyethylene glycols (PEGs) and silanes.

E.g. each ligand 9 may comprise a negatively charged functional group such as a carboxylic acid group (as the active group), in particular the carboxylic acid group being configured to allow to connect ligand 9 to nanotube 7 by means of ionic self-assembly.

According to some possible embodiments, all or some ligand 9 may correspond to m-dPEG® 37 acid, which is a long (112 atoms, 133.9 Å), methyl-capped, discrete chain length polyethylene glycol spacer.

Alternatively or additionally, all or some ligand 9 may correspond to the silane, such as (3-Aminopropyl)triethoxysilane (APTES).

In the case that one or more ligand 9 are chosen from the group of silanes, these ligands may be connected to nanotube 7 by means of silanization.

The Applicant has tested the functionalization of nanotube 7 by means of attenuated total reflection Fourier transform infrared spectroscopy. Additionally, steady state and time resolved photoluminescence experiments demonstrate that the electronic properties of photosensitizer 8 (e.g. Ce6) are preserved upon binding to the nanotube 7.

The Applicant has also studied that for scintillators 3, e.g. having Ce6 as photosensitizer 8 and m-dPEG® 37 acid as ligand 9, a broad emission of bare nanotube 7 at 450 nm is quenched, while a dominant emission from the photosensitizer 8 appears at 710 nm. I blue emission quenching suggests that part of the energy emitted from excited nanotube 7, is adsorbed by photosensitizer 8 activating its fluorescence.

Additionally, the presence of PEG as ligand 9 intensifies the emission at 710 nm, suggesting that ligand 9 further improve the scintillation properties of the functionalized nanotube 7.

The Applicant has also verified by electron paramagnetic resonance (EPR) spectroscopy in combination with 2,2,6,6-tetramethylpiperidine as a singlet oxygen specific spin trap that scintillator 3 sensitizes generation of singlet oxygen by soft X-ray. The Applicant has verified that the activation of photosensitizer 8 increases due to the presence of ligand 9.

The Applicant has found in vitro that relying on scintillators 3 at a concentration of 1 μM and radiation by electromagnetic radiation, formation of Aβ aggregates was reduced by 85%. The Applicant has also tested the efficiency of scintillators 3 in vivo on transgenic C. elegans models over expressing human Aβ.

FIG. 4 demonstrates the applicability of scintillators 3 for the treatment of diseases such as AD. The left side atomic force microscopy (AFM) image shows untreated Aβ having formed long fibril structures after 72 hours of aging in dark and on the right side one sees the result, obtained after radiation of scintillators 3 with the electromagnetic radiation (2 Gy of X-ray radiation), demonstrating the reduction of Aβ aggregates.

According to some possible embodiments, electromagnetic radiation therapy or more specifically radiotherapy, involves a plurality of scintillators 3 in combination with an electromagnetic radiation source.

According to some possible embodiments, a composite for electromagnetic radiation therapy, more specifically for radiotherapy, comprises a plurality of scintillators 3.

Such composites can e.g. be used in the treatment of degenerative diseases such as AD.

Such composite can be used so as to administer scintillators 3 into a patient.

E.g. the composite can be dissolved or suspended in a liquid prior to its administration into the patient.

According to some possible embodiments, the composite may be housed within a vial containing the composite.

According to some possible embodiments, a suspension or solution for electromagnetic radiation therapy, more specifically for radiotherapy, comprises a solvent, such as a physiological solution, and a plurality of scintillators 3 dissolved in the solvent.

Such suspensions or solutions can e.g. be used in the treatment of degenerative diseases such as AD.

Such suspension or solution can be used so as to administer scintillators 3 into a patient.

Accordingly, scintillators 3 are used in the treatment of diseases, more specifically in the treatment of degenerative diseases such as AD.

A method of producing scintillators 3 comprises the steps of:
providing one or more nanotubes 7; and
functionalizing the one or more nanotubes 7 with a plurality of single oxygen photosensitizers 8 and ligands 9.

During the step of providing, a composite of nanotube 7 is provided.

Moreover, during the step of providing, nanotubes 7 are suspended in a suspension fluid, such as a basic fluid.

E.g. nanotubes 7 may be suspended in a NaOH solution. More specifically, 30 mg of dry nanotube 7 may be suspended in 5 ml of the NaOH solution (0.4 M).

During the step of functionalizing, photosensitizers 8 are connected to nanotubes 7, e.g. by means of ionic self-assembly.

More specifically, during the step of functionalizing, photosensitizers 8 may be added to the suspension fluid. Even more specifically, during the step of functionalizing, an auxiliary suspension containing photosensitizers 8 may be added to the suspension solution.

E.g. 2.5 mL of a Ce6 solution (650 μM in NaOH 0.4 M) may be added to the suspension solution.

Additionally, during the step of functionalizing, the suspension containing nanotubes 7 and photosensitizers 8 may be centrifuged and dried obtaining an assembly of nanotubes 7 functionalized with photosensitizers 8.

In further detail, during the step of functionalizing, the assembly (e.g. 40 mg) is dispersed in a further solvent (e.g. 20 ml of Tris buffer, 100 mM, pH 7.4). The further solvent containing the assembly is then exposed to sonication and afterwards, the ligands 9 (e.g. 10 mg of m-PEG® 37-acid) are added to the further solvent.

Finally, during the step of functionalizing, the further solvent is centrifuged and dried obtaining thereby a plurality of scintillators 3.

The advantages of scintillators 3 and/or their use in electromagnetic radiation therapy according to the present invention will be clear from the foregoing description.

In particular, scintillators 3 can be used to inhibit the formation of neurotoxic Aβ oligomers through the targeted oxidation of specific amino acids that prevent aggregation of Aβ to critical sizes. Scintillators 3 allow the oxidation reaction to be obtained by exploiting reactive singlet oxygen locally produced by the interaction of scintillators 3 with the electromagnetic radiation.

Furthermore, the activation of scintillators 3 is possible also in deep tissue.

Moreover, it is possible to locally boost the production of the singlet oxygen in the proximity of Aβ monomers 5.

Additionally, activation of scintillators 3 can be based on electromagnetic radiation technologies, such as X-ray radiation technologies, developed in the context of cancer therapy.

Clearly, changes may be made to scintillators 3 and/or the use of scintillators 3 and/or the method of producing scintillators 3 as described herein without, however, departing from the scope of protection as defined in the accompanying claims.

1. Scintillator (3) for electromagnetic radiation therapy comprising a nanotube (7) being functionalized with:
a plurality of singlet oxygen photosensitizers (8) configured to form singlet oxygen (4) upon excitation by means of an electromagnetic radiation; and
a plurality of ligands (9) configured to stabilize the scintillator (3) and/or to increase solubility of the scintillator (3) within a fluid comprising water and/or to minimize interaction with biomolecules present within the fluid.

2. Scintillator according to claim 1, wherein the nanotube (7) has a length ranging between 50 nm to 1000 nm and/or a diameter ranging between 50 nm to 130 nm.

3. Scintillator according to claim 1, wherein the nanotube comprises and/or consists of magnesium silicate (Mg₃(Si₂O₅)(OH)₄) and/or comprises alternating and wrapped layers of silica and brucite.

4. Scintillator according to claim 1, wherein the singlet oxygen photosensitizers (8) are negatively charged.

5. Scintillator according to claim 4, wherein the singlet oxygen photosensitizers (8) comprise and/or consist of chlorin and/or chlorin e6 (Ce6) and/or meso-tetra(4-sulfonatophenyl)-porphyrin (H2TPPS4-) and/or porphyrin IX and/or Rose Bengal.

6. Scintillator according to claim 1, wherein each ligand (9) is chosen from the group of polyols.

7. Scintillator according to claim 1, wherein the ligand (9) comprises a negatively charged functional group.

8. Composite for electromagnetic radiation therapy comprising a plurality of scintillators (3) according to claim 1.

9. Suspension or solution for electromagnetic radiation therapy comprising a solvent and a plurality of scintillators (3) according to claim 1.

10. Method of producing scintillators according to claim 1 comprising the steps of:
providing one or more nanotubes (7); and
functionalizing the nanotubes (7) with single oxygen photosensitizers (8) and ligands (9).

11. Use of scintillators (3) according to claim 1 in the treatment of diseases.
